(19) United States
(12) Patent Application Publication
(10) Pub. No.: US 2026/0306155 A1
(43) Pub. Date: Oct. 8, 2026

(54) AUTONOMOUS ORTHOPEDICS

(71) Applicant: Kambiz Behzadi, Pleasanton, CA (US)

(72) Inventor: Kambiz Behzadi, Pleasanton, CA (US)

(21) Appl. No.: 19/533,256

(22) Filed: Feb. 8, 2026

Related U.S. Application Data
(60) Provisional application No. 63/756,278, filed on Feb. 10, 2025.
(60) Provisional application No. 63/765,699, filed on Mar. 2, 2025.

(51) Int. Cl. A61B 17/88; A61B 5/00; A61B 34/10; A61B 34/32

(52) U.S. Cl. CPC A61B 17/88; A61B 5/4504; A61B 5/7275; A61B 34/10; A61B 34/32; A61B 2034/104; A61B 2034/105

(57) ABSTRACT

A system and method for a force-adaptive robotic orthopedic surgery system digitizes force-driven tasks including bone preparation, implant insertion, and fixation by measuring and dynamically regulating mechanical interaction at bone-tool and bone-implant interfaces. Sensor-integrated instruments include multi-axis force and torque sensing and optionally vibration, acoustic, and/or electrical sensing. Processing circuitry receives sensor data and generates real-time estimates of patient-specific biomechanical properties including density, stiffness, elasticity, friction, and structural response. Physics-informed artificial intelligence models predict tissue and implant response to applied loads. Robotic actuators or drives apply controlled force, torque, vibration, and/or displacement to tools or implants. A closed-loop controller adjusts actuation based on feedback and predicted response to execute force profiles, provide force-aware decision support, and enable semi-autonomous or autonomous orthopedic assembly intraoperatively while consistently reducing fractures, micromotion, and loosening.

### CROSS REFERENCE TO RELATED APPLICATIONS

This Application claims the benefit of U.S. Provisional Application 63/765,699 filed on Mar. 2, 2025; and this Application claims the benefit of U.S. Provisional Application 63/756,278 filed on Feb. 10, 2025, the contents of each are hereby expressly incorporated by reference thereto for all purposes.

### FIELD OF THE INVENTION

The present invention relates generally to robotic-assisted orthopedic surgery and, more particularly, to force-adaptive surgical systems incorporating sensor-integrated instruments, artificial intelligence, and closed-loop control architectures for measuring, predicting, and dynamically regulating mechanical forces applied to bone and implants during orthopedic procedures.

More specifically, the invention relates to orthopedic context-specific robotic systems that digitize traditionally manual force-driven surgical tasks—including bone preparation, implant insertion, and fixation—by integrating multi-axis force sensing, real-time biomechanical modeling, physics-informed artificial intelligence, and robotic actuation to standardize and optimize surgical assembly processes.

### BACKGROUND OF THE INVENTION

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

Orthopedic surgery is fundamentally a force-dominated mechanical discipline. Unlike many medical specialties that rely primarily on visualization, precision cutting, or low-force manipulation of soft tissues, orthopedic procedures routinely involve the application of large mechanical forces to prepare bone cavities and to install implants by various modalities, including press-fit, impaction, or controlled deformation of skeletal structures.

Successful orthopedic outcomes are governed by two primary variables: 1) the spatial position and alignment of implants relative to patient anatomy; and 2) the magnitude, direction, rate, and cumulative application of mechanical forces used during bone preparation and implant installation.

While substantial technological development has occurred in robotic systems for improving implant positioning, alignment, and geometric accuracy, existing robotic platforms largely neglect the management, sensing, and adaptive control of surgical force.

Contemporary orthopedic robotic systems primarily automate spatial navigation using imaging, optical tracking, and preoperative planning models. These systems assist surgeons in achieving desired implant placement geometries but leave all force-driven tasks, including reaming, broaching, implant impaction, and fixation—to manual execution using mallets, broaches, reamers, and other primitive mechanical tools.

As a result, the most critical determinants of implant stability and long-term success remain dependent on qualitative surgeon judgment derived from tactile sensation, auditory cues, and experiential intuition.

Orthopedic assembly processes stand in stark contrast to virtually all other mechanical industries—such as aerospace, automotive manufacturing, structural engineering, and precision machinery—where materials, geometry, and assembly forces are rigorously standardized, measured, and controlled under formal engineering specifications.

In orthopedics, while implant materials and geometric designs are extensively regulated and standardized, there exists no comparable standardization of the mechanical assembly process by which implants are installed into bone.

This absence of force standardization contributes directly to variability in surgical technique, inconsistent outcomes, and a high incidence of complications such as: intraoperative fractures; insufficient primary stability; excessive micromotion; and aseptic loosening; implant malalignment due to uncontrolled force vectors.

Clinical data consistently demonstrate that a substantial portion of orthopedic failures arise not from implant design defects or positional inaccuracies, but from improper force application during installation.

Moreover, orthopedic surgical environments are characterized by highly variable biomechanical properties, including differences in bone mineral density, elastic modulus, cortical thickness, trabecular structure, soft tissue resistance, and pathological defects. These variables produce stochastic mechanical responses to applied force that cannot be reliably predicted by preoperative imaging alone.

Consequently, surgeons rely heavily on tacit sensorimotor skills developed over years of experience to judge: how much bone to remove; when adequate press-fit has been achieved; how hard to strike/impact an implant; and when to cease force application.

This form of experiential knowledge remains largely unwritten, unquantified, and non-standardized.

Attempts to automate orthopedic surgery using conventional robotic paradigms have been limited by this fundamental gap. While positional automation is relatively straightforward using spatial sensors, the automation of force-based sensorimotor tasks requires real-time physical interaction modeling, adaptive control, and intelligent decision-making based on dynamic mechanical feedback.

Existing surgical robots lack the capability to sense high-magnitude orthopedic forces, to model bone-implant mechanical interactions in real time, or to dynamically adjust force application in response to patient-specific biomechanical conditions.

Accordingly, there exists a critical unmet need for orthopedic robotic systems capable of quantifying, predicting, and adaptively controlling surgical force in real time, thereby transforming orthopedic assembly from a qualitative artisanal process into a standardized, data-driven, and optimized engineering procedure.

### BRIEF SUMMARY OF THE INVENTION

Disclosed are a system and method for providing orthopedic robotic systems capable of quantifying, predicting, and adaptively controlling surgical force in real time, thereby transforming orthopedic assembly from a qualitative artisanal process into a standardized, data-driven, and optimized engineering procedure.

Some embodiments of the present invention may provide a force-adaptive orthopedic robotic system that integrates sensorized surgical instruments, real-time biomechanical estimation, artificial intelligence, and closed-loop robotic actuation to dynamically regulate mechanical forces applied during orthopedic procedures.

An embodiment of a system includes: one or more orthopedic instruments incorporating multi-axis force, torque, vibration, acoustic, and/or electrical sensing elements configured to measure mechanical interactions at bone-tool and bone-implant interfaces; one or more processing units configured to receive sensor data and generate real-time estimates of biomechanical properties including bone density, stiffness, elasticity, frictional resistance, and structural response; one or more artificial intelligence models, including physics-informed neural networks trained on biomechanical simulations and surgical data, configured to predict tissue response to applied force; one or more robotic actuators or electromechanical drive systems configured to apply controlled force, torque, vibration, or displacement to surgical instruments or implants; and a closed-loop control architecture configured to dynamically adjust force application based on real-time sensor feedback and predicted biomechanical response.

Some embodiments of the system convert traditionally manual, qualitative force-driven surgical tasks into quantitative, adaptive, and standardized mechanical processes.

In certain embodiments, the system digitizes conventional orthopedic tools such as reamers, broaches, impactors, and insertion devices by integrating sensing elements and control systems, transforming them into electromechanical instruments capable of force measurement and adaptive regulation.

In further embodiments, the artificial intelligence layer predicts patient-specific mechanical responses using physics-based modeling, enabling personalized force profiles that optimize implant fixation and minimize tissue damage.

Some embodiments of the present invention may further support progressive levels of autonomy, including: a) force-aware surgical decision support; b) semi-autonomous force execution under surgeon supervision; and c) fully autonomous execution of defined orthopedic assembly tasks.

By standardizing surgical force application in real time, the invention reduces variability, improves implant stability, minimizes complications, and establishes a foundation for autonomous orthopedic surgery.

A force-adaptive autonomous orthopedic surgical system including: at least one orthopedic instrument configured to mechanically engage bone or an implant; one or more sensors integrated with the instrument and configured to generate real-time data representative of mechanical interaction between the instrument, bone, and implant, including at least one of force, torque, vibration, acoustic response, deformation, or electrical response; a processing system configured to estimate biomechanical properties of the bone and bone-implant interface from the real-time data; an artificial intelligence subsystem comprising one or more physics-informed models trained on biomechanical simulations and operative data, the subsystem configured to predict mechanical response of skeletal tissue to applied force; a robotic actuation system configured to apply mechanical force to the instrument or implant; and a closed-loop control architecture configured to dynamically regulate magnitude, direction, rate, and termination of the applied mechanical force based on the real-time data and predicted biomechanical response, wherein the system autonomously optimizes orthopedic assembly and fixation through predictive force-adaptive control.

A method of performing orthopedic surgery using predictive force-adaptive control, including: mechanically engaging bone or an implant with a sensor-integrated orthopedic instrument; measuring real-time mechanical interaction data between the instrument and skeletal tissue; estimating biomechanical properties of the tissue from the mechanical interaction data; predicting tissue response to applied force using a physics-informed artificial intelligence model; dynamically applying mechanical force using a robotic actuator based on the predicted tissue response; and modulating or terminating force application when predicted biomechanical stability or safety thresholds are achieved, and optimizing autonomously implant fixation and bone preparation.

A biomechanical artificial intelligence control system for regulating orthopedic surgical force, including: one or more neural network models constrained by physical laws of tissue deformation, fracture mechanics, and contact dynamics; training data comprising biomechanical simulations and real-time surgical sensor data; a real-time inference engine configured to predict skeletal tissue response to incremental force application; and an output interface configured to generate force-control commands for robotic surgical actuators, wherein the system anticipates biomechanical outcomes and governs adaptive force regulation during orthopedic procedures.

An autonomous orthopedic surgical platform including: multiple force-adaptive robotic surgical systems operating across clinical sites; a data aggregation network configured to collect biomechanical interaction data from the systems; a centralized or federated learning architecture configured to update physics-informed artificial intelligence models using the aggregated data; and a set of deployment mechanisms for distributing updated models to the robotic systems; wherein predictive force control performance improves continuously with accumulated surgical experience.

An autonomous robotic orthopedic system configured to perform force-driven surgical tasks without manual force input, including: sensorized surgical instruments configured to measure bone-tool mechanical interaction; a predictive biomechanical intelligence engine configured to forecast mechanical outcomes of force application; robotic actuators configured to apply controlled mechanical force to the instruments; and a closed-loop controller configured to autonomously execute bone preparation, implant insertion, and fixation optimization within biomechanical safety envelopes.

Any of the embodiments described herein may be used alone or together with one another in any combination. Inventions encompassed within this specification may also include embodiments that are only partially mentioned or alluded to or are not mentioned or alluded to at all in this brief summary or in the abstract. Although various embodiments of the invention may have been motivated by various deficiencies with the prior art, which may be discussed or alluded to in one or more places in the specification, the embodiments of the invention do not necessarily address any of these deficiencies. In other words, different embodiments of the invention may address different deficiencies that may be discussed in the specification. Some embodiments may only partially address some deficiencies or just one deficiency that may be discussed in the specification, and some embodiments may not address any of these deficiencies.

Other features, benefits, and advantages of the present invention will be apparent upon a review of the present disclosure, including the specification, drawings, and claims.

### BRIEF DESCRIPTION OF THE DRAWINGS

The accompanying figures, in which like reference numerals refer to identical or functionally-similar elements throughout the separate views and which are incorporated in and form a part of the specification, further illustrate the present invention and, together with the detailed description of the invention, serve to explain the principles of the present invention.

FIG. 1 illustrates an Evolution of Technology over time and its relationship with tools currently used in Orthopedic Surgery;

FIG. 2 illustrates a multi-disciplinary nature of the total hip replacement procedure and how current robotic systems appear to focus only on alignment and position of the implant, ignoring the press-fit and bone sizing aspects of the surgery, which relate to management of force;

FIG. 3 illustrates an Analogy of total hip replacement with current sport of MMA, where a variety of different skills are required for success;

FIG. 4 illustrate Aspects of total hip replacement surgery that are related to application of force. An attempt to segregate these sections and convert them from qualitative to quantitative; and finally to routinize and automate;

FIG. 5 illustrate Causes of failure in total hip replacement associated with management of force application (assembly technique), materials and design;

FIG. 6 illustrates an evolution of AI from text recognition to content creation to physical AI;

FIG. 7 illustrates an Application of physical AI to the foundational physical system of OCSI (orthopedic context specific instruments) to create OCSAI (orthopedic context specific AI);

FIG. 8 illustrates an Applicability of current robotic systems to “low force” orthopedic procedures, where alignment and position is paramount and where there is little need for application of force based on experiential tacit knowledge;

FIG. 9 illustrates an evolution of AI from perception to generative to agentic to physical. Also, the evolution of OCSAI and its many species, based on the original foundational model of OCSI;

FIG. 10 illustrates force related aspects of orthopedic surgery, qualitatively and subjectively assessed by the surgeon: (How much should I ream? How much should I broach? How hard should I hit? When should I stop impacting? Is the direction of my force correct?”;

FIG. 11 illustrates an algorithm for Automatic Prosthesis Installation Machine (APIM) for best fixation short of fracture;

FIG. 12 illustrates an APIM or AI-PIM (automatic intelligent prosthesis installation machine) is a perfect foundational base for Machine learning to create neural networks that can be trained on biomechanical simulations;

FIG. 13 illustrates an AI-PIM as a model for closed-loop feedback mechanism;

FIG. 14 illustrates A schematic representation of integrating sensors into surgical instruments to create ESSOB (electronic signature sizing of bone) a species of (orthopedic context specific instruments) OCSI. To create tools that can assess the implant bone condition quantitatively instead of relying on the surgeon's qualitative sensorimotor, auditory and tactile senses.

FIG. 15 illustrates a variation of FIG. 14;

FIG. 16 illustrates electronic analogue of the mechanical system using a reamer to cut bone;

FIG. 17 illustrates electronic analogue of mechanical systems of reaming and broaching with addition of Kalman filter to increase precision;

FIG. 18 illustrates various ways to assess the frictional forces at the implant bone interface, using direct and indirect measurement techniques; and

FIG. 19 illustrates a Schematic of a sensor integrated acetabular reamer, connected to a computer to sense the frictional forces and the quality of contact between the bone and implant.

### DETAILED DESCRIPTION OF THE INVENTION

Embodiments of the present invention provide a system and method for providing robotic-assisted orthopedic surgery, particularly an AI-powered robotic system that measures, adapts and applies a more reliably and more optimal force during surgery to reduce variability and improve outcomes. The following description is presented to enable one of ordinary skill in the art to make and use the invention and is provided in the context of a patent application and its requirements.

Various modifications to the preferred embodiment and the generic principles and features described herein will be readily apparent to those skilled in the art. Thus, the present invention is not intended to be limited to the embodiment shown but is to be accorded the widest scope consistent with the principles and features described herein.

### Definitions

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The following definitions apply to some of the aspects described with respect to some embodiments of the invention. These definitions may likewise be expanded upon herein.

As used herein, the term “or” includes “and/or” and the term “and/or” includes any and all combinations of one or more of the associated listed items. Expressions such as “at least one of,” when preceding a list of elements, modify the entire list of elements and do not modify the individual elements of the list.

As used herein, the singular terms “a,” “an,” and “the” include plural referents unless the context clearly dictates otherwise. Thus, for example, reference to an object can include multiple objects unless the context clearly dictates otherwise.

Also, as used in the description herein and throughout the claims that follow, the meaning of “in” includes “in” and “on” unless the context clearly dictates otherwise. It will be understood that when an element is referred to as being “on” another element, it can be directly on the other element or intervening elements may be present therebetween. In contrast, when an element is referred to as being “directly on” another element, there are no intervening elements present.

As used herein, the term “set” refers to a collection of one or more objects. Thus, for example, a set of objects can include a single object or multiple objects. Objects of a set also can be referred to as members of the set. Objects of a set can be the same or different. In some instances, objects of a set can share one or more common properties.

As used herein, the term “adjacent” refers to being near or adjoining. Adjacent objects can be spaced apart from one another or can be in actual or direct contact with one another. In some instances, adjacent objects can be coupled to one another or can be formed integrally with one another.

As used herein, the terms “connect,” “connected,” and “connecting” refer to a direct attachment or link. Connected objects have no or no substantial intermediary object or set of objects, as the context indicates.

As used herein, the terms “couple,” “coupled,” and “coupling” refer to an operational connection or linking. Coupled objects can be directly connected to one another or can be indirectly connected to one another, such as via an intermediary set of objects.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%.

As used herein, the terms “substantially” and “substantial” refer to a considerable degree or extent. When used in conjunction with an event or circumstance, the terms can refer to instances in which the event or circumstance occurs precisely as well as instances in which the event or circumstance occurs to a close approximation, such as accounting for typical tolerance levels or variability of the embodiments described herein.

As used herein, the terms “optional” and “optionally” mean that the subsequently described event or circumstance may or may not occur and that the description includes instances where the event or circumstance occurs and instances in which it does not.

As used herein, the term “size” refers to a characteristic dimension of an object. Thus, for example, a size of an object that is spherical can refer to a diameter of the object. In the case of an object that is non-spherical, a size of the non-spherical object can refer to a diameter of a corresponding spherical object, where the corresponding spherical object exhibits or has a particular set of derivable or measurable properties that are substantially the same as those of the non-spherical object. Thus, for example, a size of a non-spherical object can refer to a diameter of a corresponding spherical object that exhibits light scattering or other properties that are substantially the same as those of the non-spherical object. Alternatively, or in conjunction, a size of a non-spherical object can refer to an average of various orthogonal dimensions of the object. Thus, for example, a size of an object that is a spheroidal can refer to an average of a major axis and a minor axis of the object. When referring to a set of objects as having a particular size, it is contemplated that the objects can have a distribution of sizes around the particular size. Thus, as used herein, a size of a set of objects can refer to a typical size of a distribution of sizes, such as an average size, a median size, or a peak size.

Orthopedic surgery involves use of press fit arthroplasty. In this process the bone cavity is prepared approximately 1 mm smaller than the implant, and the implant is impacted into place with dynamic impacts. What can be useful in many contexts is an AI-powered robotic system that measures, adapts and applies optimal force during surgery to reduce variability and improve outcomes.

Which brings up an environment that may be addressed by one or more embodiments disclosed or reasonably suggested to a person having ordinary skill in the art: how does one take a sensorimotor skill that is experiential and has no data; collect data on it, routinize it, and then automate it to make surgeries safer for patients?

Some embodiments may include a focus on the first step of this process. To assist surgeons in making better decisions, the solution framework includes tools that allow surgeons to make decisions based on a quantitative process (numbers) rather than a qualitative process (tactile feel). Some solutions have digitized basic cutting and impacting tools (reamers, broaches and mallets) by integrating sensors and simple control algorithms.

These tools may include one or more of the following: 1. Electronic Signature Sizing of Bone (ESSOB) a sensor-based system for bone cavity sizing based on elasticity and density; 2. Automatic Prosthesis Installation Machine (APIM), a sensor-based impaction system that modulates impactions forces reducing fracture risk; and 3. Vibratory Insertion of Orthopedic Implants (VIOI) a vibratory insertion method for reducing bone damage while optimizing implant seating.

With the advent of machine learning, embodied systems and sensorimotor learning, tools and solutions may consider these electromechanical tools as a platform for development of first AI-driven physical system in orthopedic surgery.

By integrating Machine learning, Accelerated Computing and Robotic actuators into these tools, there has to be a conception of specialized robots that can not only sense but also think and interact with their human skeletal environment.

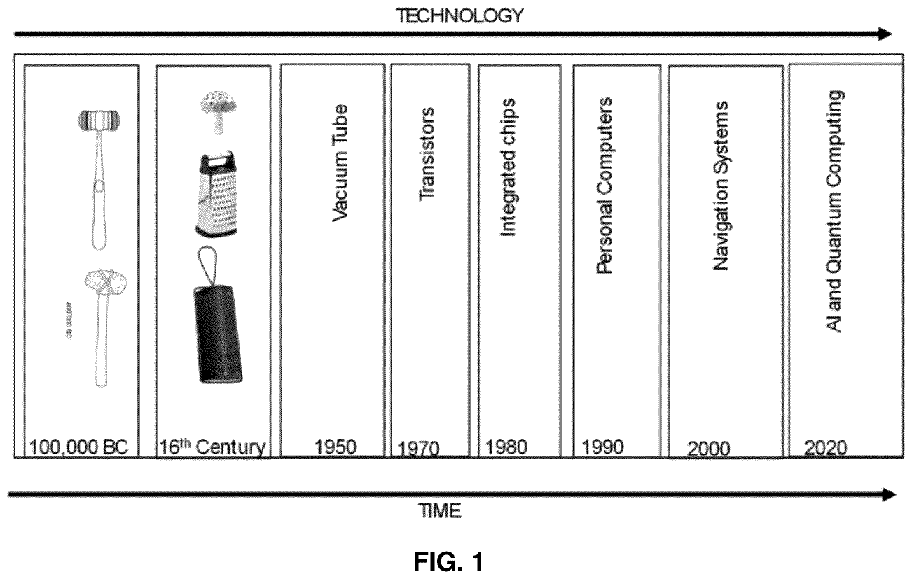

FIG. 1 illustrates a historical progression of technological paradigms over time, depicting the evolution from manual tools through electromechanical systems, solid-state electronics, integrated computing, and navigation systems, culminating in modern intelligent and autonomous systems. The figure is intended to contextualize the disclosed orthopedic context-specific artificial intelligence systems within a broader technological evolution.

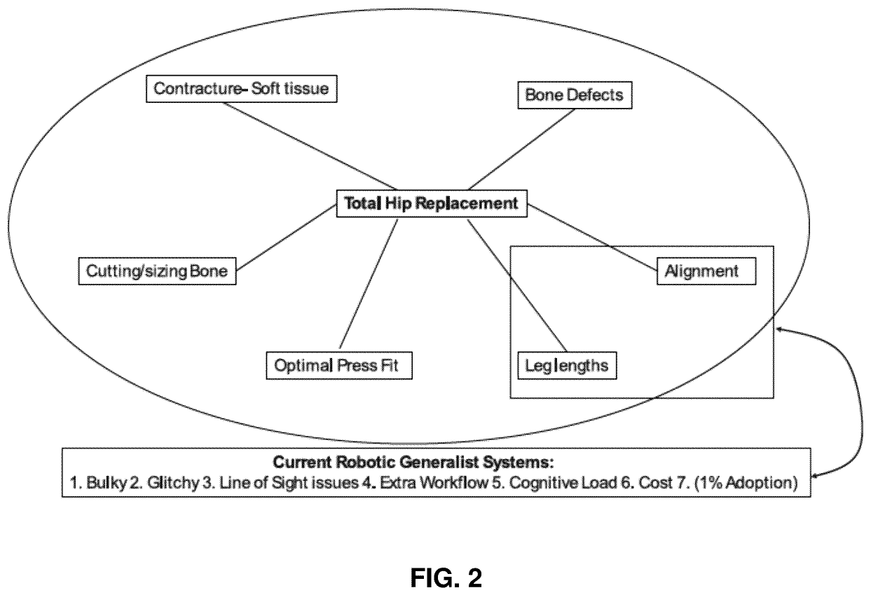

FIG. 2 illustrates representative challenges encountered during a total hip replacement procedure, including bone defects, soft-tissue contracture, implant alignment, press-fit optimization, leg-length restoration, and bone cutting and sizing, alongside limitations of existing general-purpose robotic surgical systems.

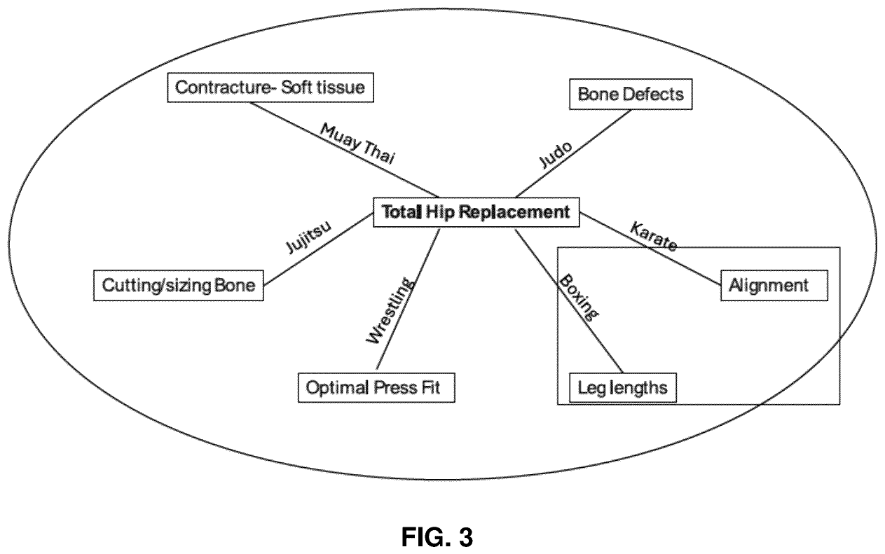

FIG. 3 further illustrates procedural variables relevant to orthopedic joint replacement, including anatomical variability, alignment constraints, press-fit requirements, and bone preparation steps, emphasizing the complexity of surgical decision-making in orthopedic procedures.

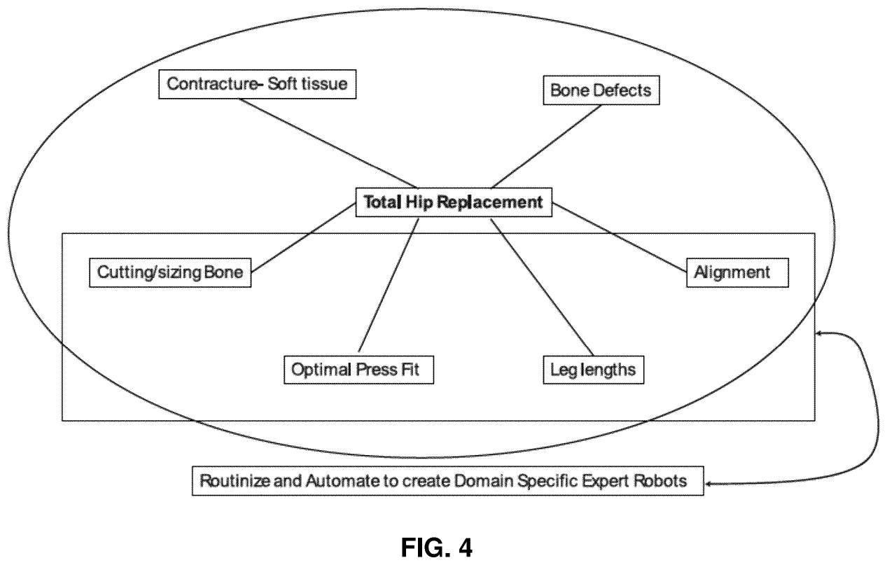

FIG. 4 illustrates a conceptual transition from manual and generalized robotic assistance toward routinized and automated orthopedic workflows using domain-specific expert robotic systems configured to address the procedural variables shown in FIGS. 2-3.

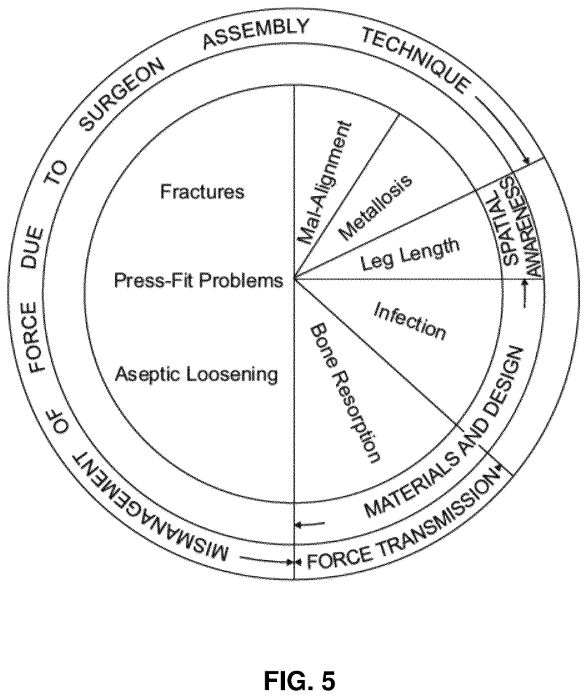

FIG. 5 illustrates a systems-level view of orthopedic implant failure modes and clinical complications, including fractures, malalignment, metallosis, leg-length discrepancy, infection, bone resorption, press-fit problems, and aseptic loosening, arranged relative to force transmission, material design, and surgical assembly processes.

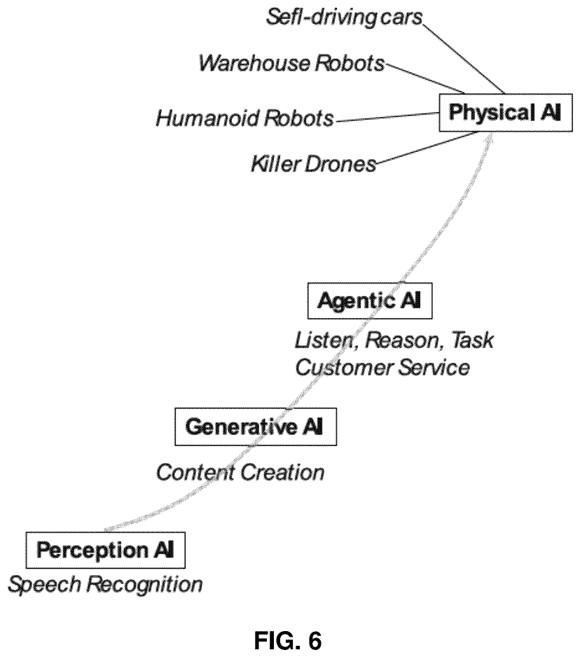

FIG. 6 illustrates a taxonomy of artificial intelligence modalities, including perception AI, generative AI, agentic AI, and physical AI, and associates each modality with representative applications ranging from speech recognition and content creation to autonomous physical systems.

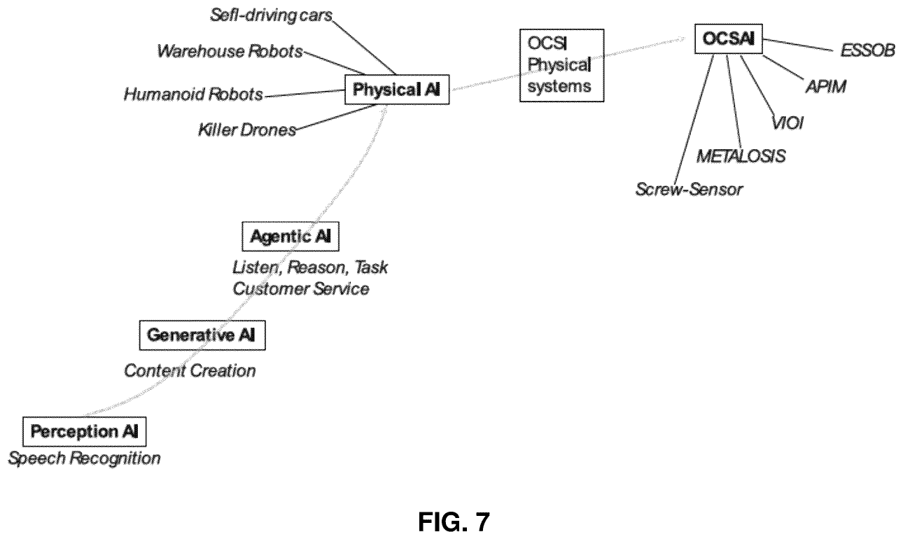

FIG. 7 illustrates an extension of the artificial intelligence taxonomy of FIG. 6, incorporating orthopedic context-specific AI systems, including physical orthopedic systems, sensorized implants, force-aware instrumentation, and physiological monitoring subsystems.

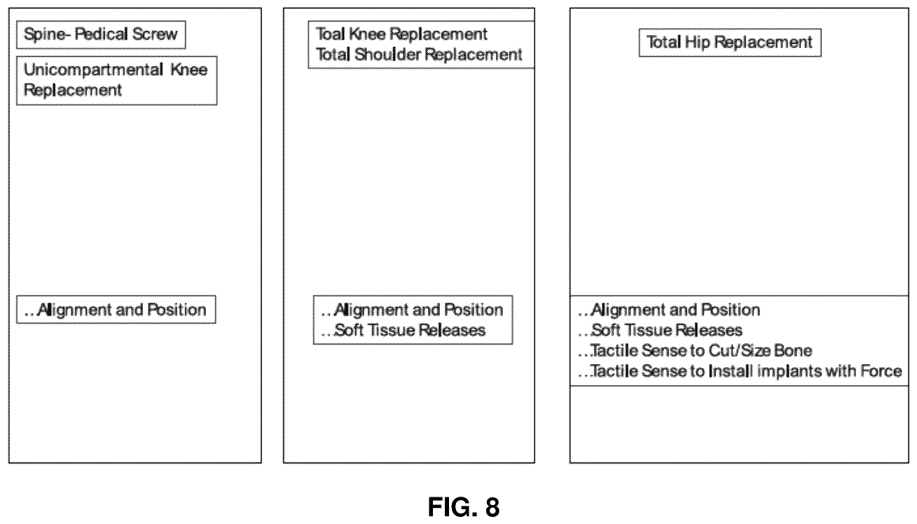

FIG. 8 illustrates representative orthopedic procedures benefiting from context-specific physical AI, including unicompartmental knee replacement, total knee replacement, total hip replacement, shoulder replacement, spinal fixation, and associated alignment, soft-tissue management, and force-controlled implantation tasks.

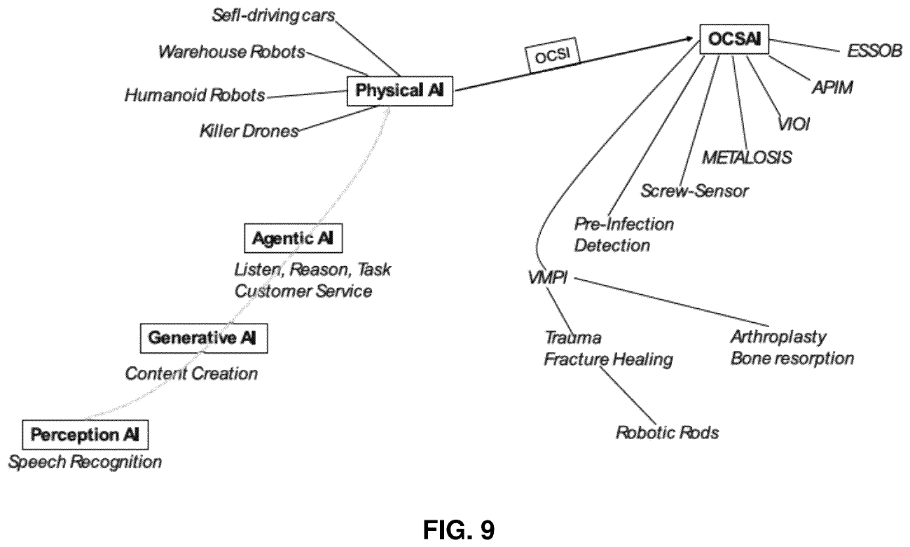

FIG. 9 illustrates an expanded orthopedic application landscape integrating artificial intelligence modalities with orthopedic-specific sensing, monitoring, and actuation systems for arthroplasty, trauma, fracture healing, bone resorption monitoring, and pre-infection detection.

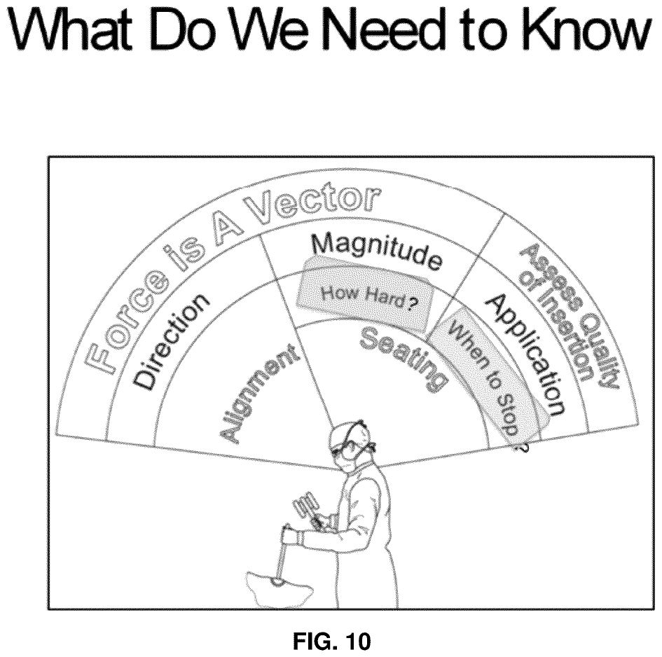

FIG. 10 illustrates a conceptual framework in which applied forces during orthopedic procedures are treated as vector quantities having magnitude, direction, alignment, timing, and application constraints, emphasizing the informational requirements necessary for controlled implantation.

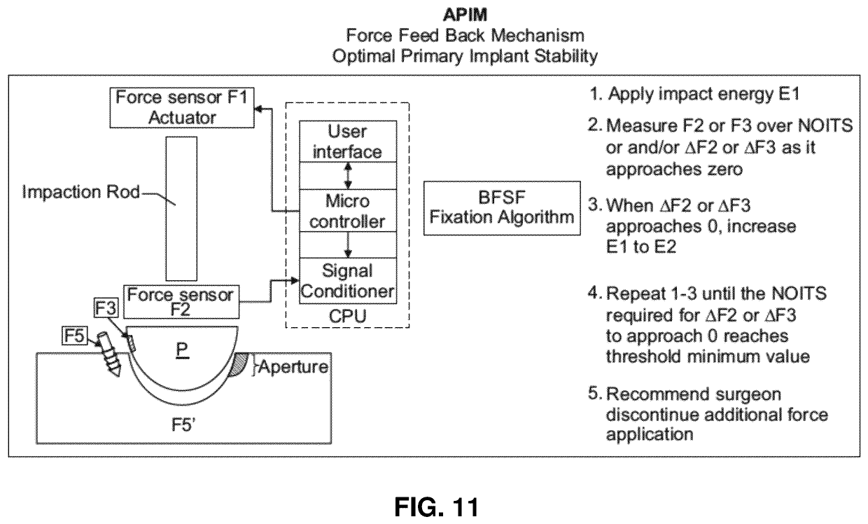

FIG. 11 illustrates an embodiment of an active press-fit implantation mechanism (APIM), including force sensors, actuators, signal conditioning electronics, a controller, and a fixation algorithm configured to regulate force application during implant seating.

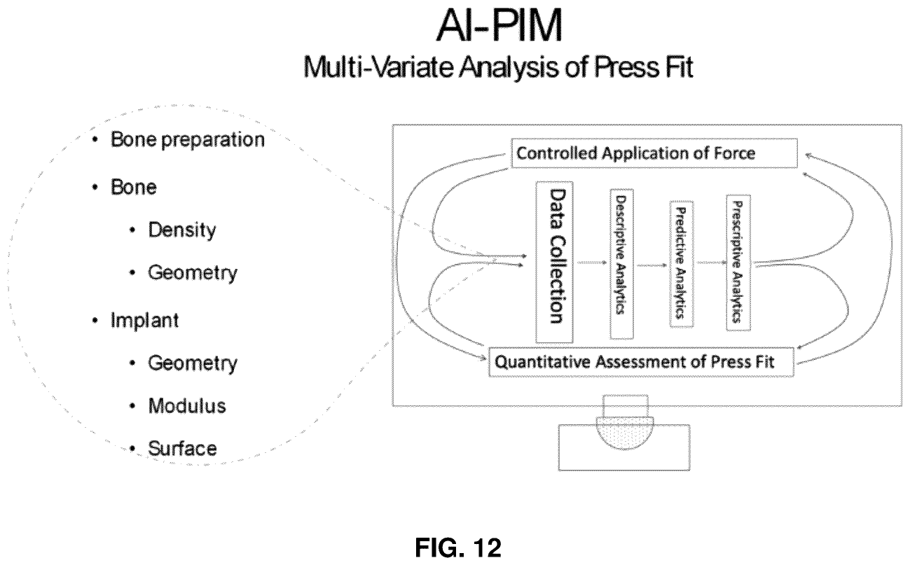

FIG. 12 illustrates an artificial-intelligence-based press-fit inference model (AI-PIM) configured to perform multivariate analysis of bone properties, implant geometry, surface characteristics, and applied force data to quantitatively assess press-fit conditions.

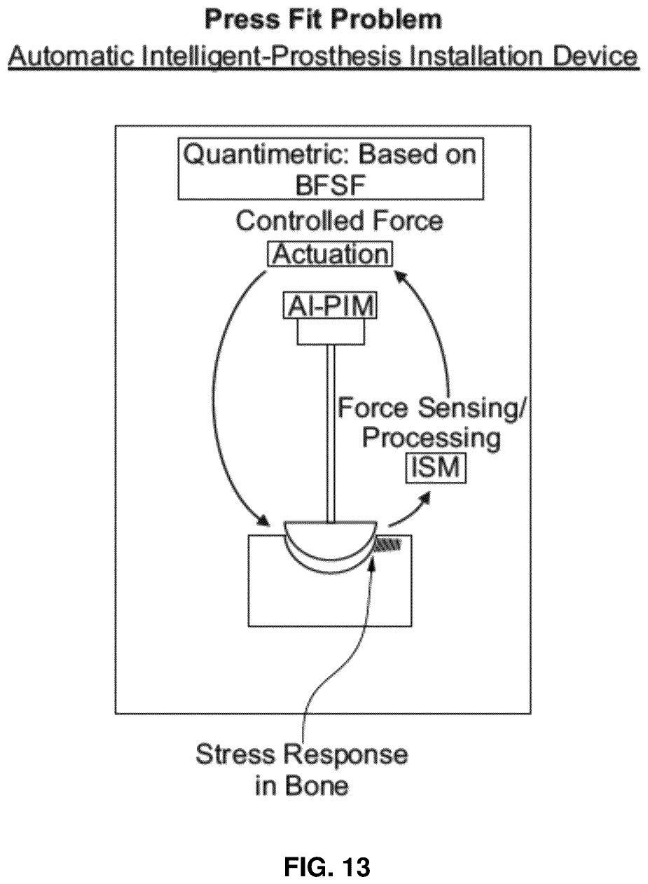

FIG. 13 illustrates an automatic intelligent prosthesis installation device implementing the AI-PIM of FIG. 12 in a closed-loop system, wherein sensed force responses from bone are used to regulate controlled actuation during implantation.

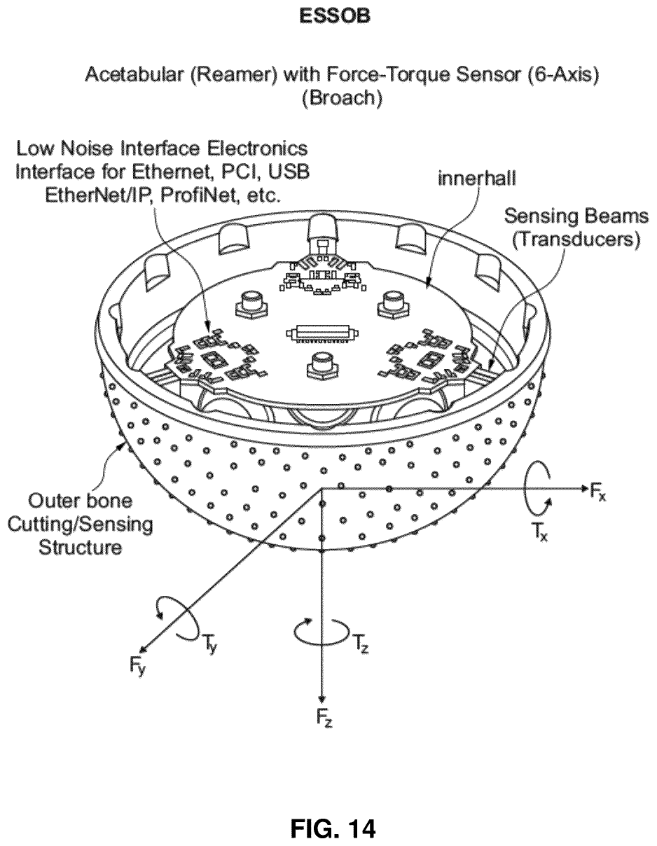

FIG. 14 illustrates an embodiment of an embedded sensing structural orthopedic body (ESSOB), such as an acetabular reamer or broach, incorporating multi-axis force and torque sensing elements, internal electronics, and data interfaces.

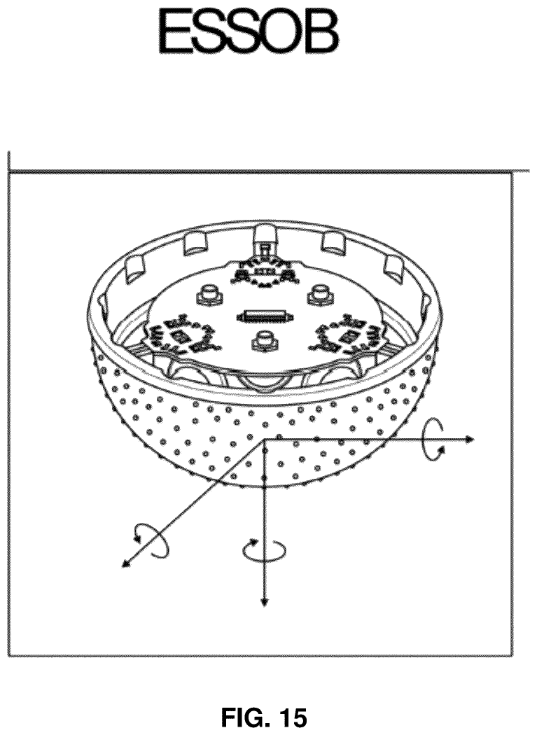

FIG. 15 illustrates an alternate perspective view of the ESSOB of FIG. 14, further showing distributed sensing beams and rotational degrees of freedom for detecting applied loads during bone preparation or implant seating.

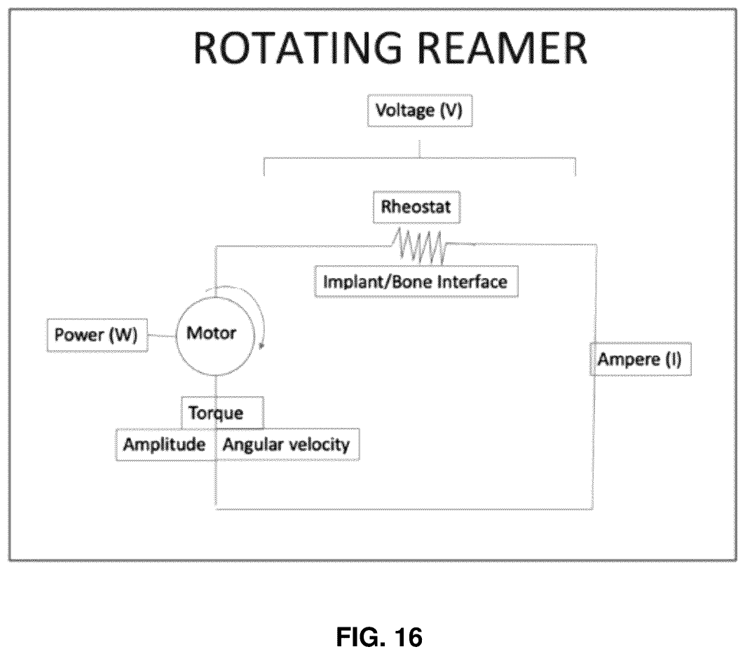

FIG. 16 illustrates a rotating reamer system model, wherein electrical inputs such as voltage and current are correlated with mechanical outputs including torque, angular velocity, and power at a bone-implant interface.

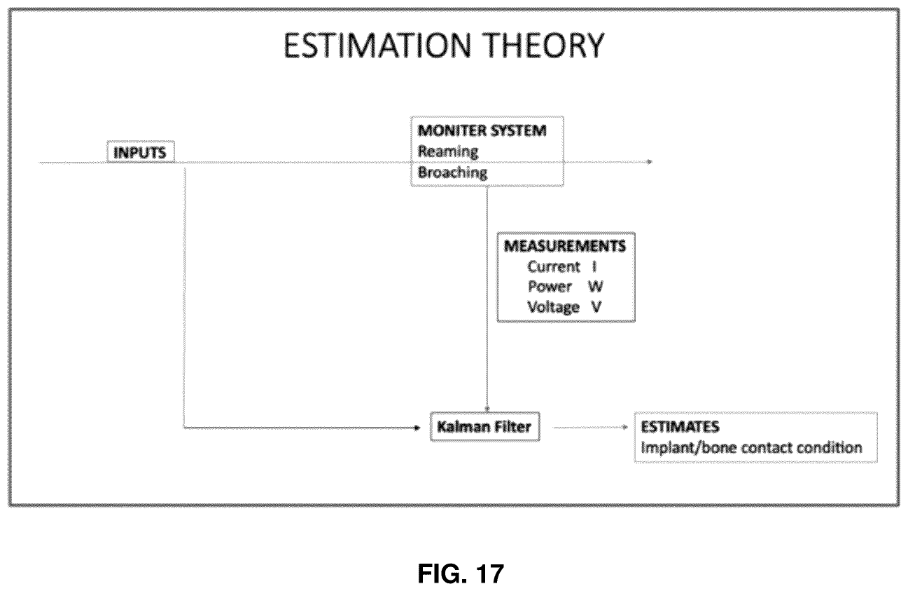

FIG. 17 illustrates an estimation framework in which monitored system inputs and electrical measurements are processed through estimation algorithms, including Kalman filtering, to infer implant-bone contact conditions.

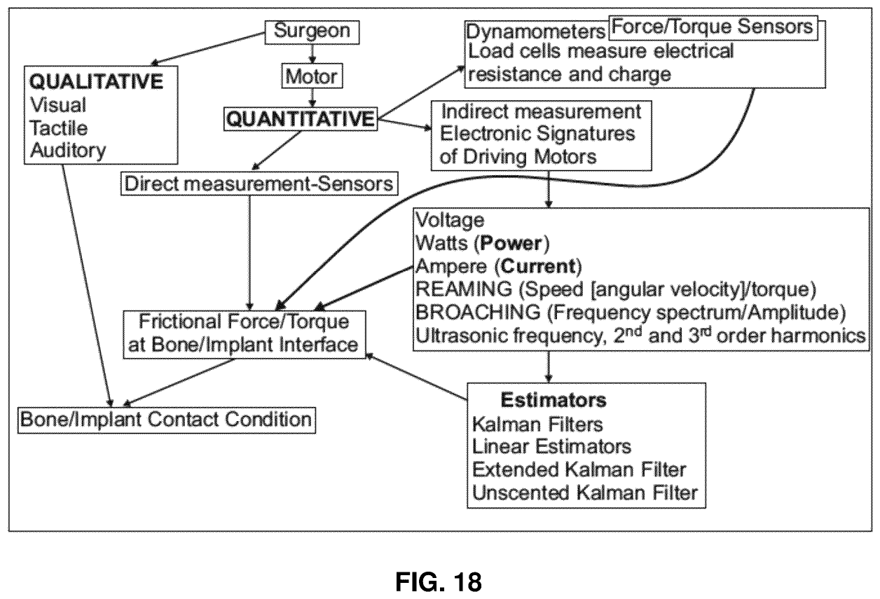

FIG. 18 illustrates a combined qualitative and quantitative sensing architecture integrating surgeon input, direct force sensing, indirect electrical measurements, and estimator models to determine frictional force and torque at a bone-implant interface.

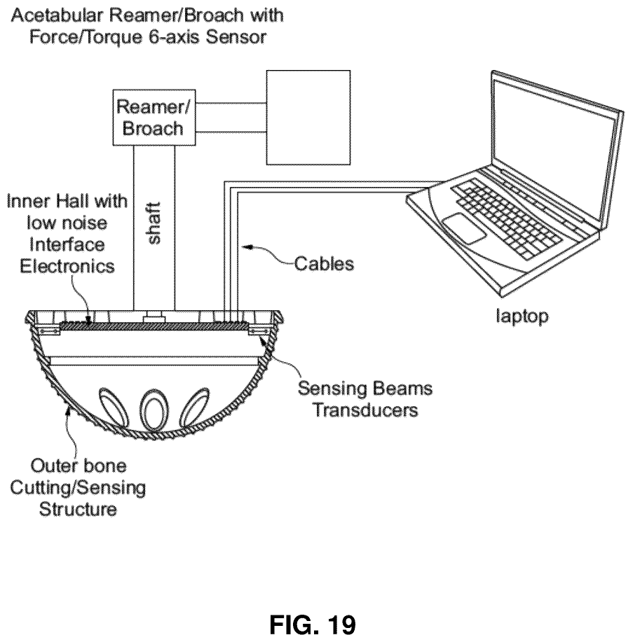

FIG. 19 illustrates a system-level embodiment including a sensorized acetabular reamer or broach coupled via cables to external computing equipment, illustrating signal transmission pathways between embedded sensors and processing systems.

### System Architecture Overview

In general, the force-adaptive orthopedic robotic system operates as a closed-loop cyber-physical control architecture comprising sensing, estimation, prediction, and actuation subsystems.

### Sensorized Orthopedic Instruments

One or more surgical instruments are equipped with integrated sensing elements configured to measure mechanical interactions during bone preparation and implant insertion.

These sensors may include, without limitation: multi-axis force and torque sensors; strain gauges; piezoelectric sensors; accelerometers and vibration sensors; acoustic emission sensors; and electrical impedance and motor current sensors.

The sensors are positioned to capture real-time data corresponding to cutting resistance, interface friction, deformation response, impact forces, vibratory behavior, and contact conditions.

### Biomechanical Estimation Layer

Sensor data are transmitted to a processing unit configured to estimate biomechanical parameters such as: effective bone stiffness and elasticity; resistance profiles during cutting and insertion; contact area and interference fit magnitude; and dynamic response to impact or vibration.

Estimation may be performed using signal processing algorithms, observers, Kalman filtering techniques, and real-time system identification methods.

### Artificial Intelligence Prediction Layer

An artificial intelligence subsystem receives estimated biomechanical parameters and predicts tissue response to applied force.

In preferred embodiments, the AI subsystem comprises physics-informed neural networks trained using biomechanical simulations combined with empirical surgical data.

These models generate real-time predictions of: deformation behavior; fracture thresholds; optimal press-fit ranges; force-displacement relationships; and risk of mechanical failure.

The AI thereby enables patient-specific adaptive force control.

### Closed-Loop Robotic Actuation

Robotic actuators or electromechanical drive systems apply controlled force, torque, vibration, or displacement to surgical instruments or implants.

Control signals are continuously updated based on sensor feedback and AI predictions to: increase or decrease applied force; adjust directionality of force vectors; modulate insertion speed; and terminate force application at optimal stability thresholds.

The system thereby executes a closed-loop adaptive force regulation process throughout surgical assembly.

### Sensor-Integrated Orthopedic Control System Instruments (OCSI) as the Physical Foundation of Autonomous Orthopedics (AO)

The force-adaptive robotic systems described herein are constructed upon a class of sensor-integrated electromechanical surgical instruments collectively referred to as Orthopedic Context-Specific Instruments (OCSI).

OCSI constitute the physical sensing and actuation substrate through which Autonomous Orthopedics (AO), also referred to as Autonomous Orthopedic Systems (Aut-O-Sys), is implemented.

In particular, OCSI provides the real-time mechanical interaction data and force-application capability necessary for closed-loop artificial intelligence control. AO systems comprise the integration of OCSI instruments with artificial intelligence models, biomechanical estimation algorithms, and robotic control architectures to achieve adaptive and autonomous force regulation during orthopedic surgery.

Thus: OCSI may represent the instrument-level electromechanical embodiments; and AO represents the system-level autonomous robotic embodiment built upon OCSI.

Each OCSI embodiment may operate in a passive measurement mode, a semi-autonomous adaptive mode, or a fully autonomous force-execution mode under AO control.

The following sections describe representative OCSI embodiments that collectively enable AO.

### Electronic Signature Sizing of Bone (ESSOB)

In one embodiment, an OCSI instrument comprises an Electronic Signature Sizing of Bone (ESSOB) system configured to quantitatively characterize bone cavity geometry and mechanical properties during preparation.

ESSOB may be implemented in reamers, broaches, cutting tools, or expandable sizing devices incorporating integrated sensing elements positioned to measure multi-axis forces, torques, vibrations, acoustic emissions, and electrical parameters at the bone interface.

During bone preparation, ESSOB instruments generate a real-time electronic signature corresponding to: cutting resistance; elastic deformation response; frictional engagement; density-dependent stiffness variations; and contact surface evolution.

The electronic signature is processed to estimate one or more of: effective bone modulus; interference fit capacity; cavity conformity; and risk thresholds for fracture or excessive deformation.

Under AO control, ESSOB output is supplied to artificial intelligence models that dynamically determine one or more of: optimal cavity size; termination of cutting or broaching; and real-time force modulation, thereby converting tactile sizing into quantitative adaptive control.

ESSOB may operate as a standalone quantitative instrument or as a closed-loop autonomous bone preparation module under AO.

### Automatic Prosthesis Installation Machine (APIM)

In another embodiment, an OCSI instrument comprises an Automatic Prosthesis Installation Machine (APIM) configured to control implant insertion forces with precision.

APIM may include: force-sensing impact rods or insertion shafts; actuator systems capable of controlled impaction, continuous press-fit, or vibratory insertion; processing units implementing fixation optimization algorithms; APIM continuously measures one or more of: insertion force magnitude; force-displacement response; implant stability progression; and dynamic resistance behavior.

From these data, biomechanical estimators and AI models determine optimal press-fit conditions corresponding to sufficient primary stability without exceeding fracture or damage thresholds.

Under AO control, APIM dynamically modulates one or more of: impact energy; insertion speed; applied force vectors; and termination of insertion, to achieve reproducible optimal fixation.

APIM thereby replaces manual mallet-based impaction with controlled electromechanical force application governed by real-time biomechanical intelligence.

### Vibratory Insertion of Orthopedic Implants (VIOI)

In further embodiments, OCSI instruments implement Vibratory Insertion of Orthopedic Implants (VIOI), wherein controlled vibrational energy is applied to facilitate implant seating.

VIOI systems may include: piezoelectric or electromechanical vibratory actuators; force and acceleration sensors; and frequency modulation controllers.

Vibration parameters may be dynamically adjusted by AO based on bone response to: reduce peak insertion forces; improve alignment control; minimize microfracture risk; and optimize interface conformity.

VIOI may operate independently or in combination with APIM impaction control under AO supervision.

### Sensorized Retrofitting of Conventional Orthopedic Tools

In certain embodiments, existing orthopedic instruments may be retrofitted with sensing and actuation modules to form OCSI instruments.

Such retrofitting may include: attachment of multi-axis force sensors; integration of motor current monitoring; vibration and acoustic transducers; and microcontroller-based signal processing.

Thereby, conventional reamers, broaches, impactors, drills, and insertion handles are transformed into intelligent electromechanical instruments capable of real-time biomechanical measurement and adaptive force control.

These retrofitted OCSI instruments interface directly with AO processing systems to enable closed-loop force regulation without requiring complete replacement of surgical toolsets.

### Progressive Autonomy Enabled by OCSI Under AO Control

Each OCSI embodiment may function across progressive levels of automation, including: 1. quantitative sensing and real-time feedback to surgeons; 2. AI-assisted force recommendations; 3. semi-autonomous adaptive force execution; and 4. fully autonomous surgical force application under AO.

Accordingly, AO systems utilize OCSI instruments as the physical execution layer while artificial intelligence governs force strategy, optimization, and safety control.

Through this layered architecture, AO converts orthopedic surgery from manual force craftsmanship into a standardized, adaptive, and autonomous engineering process.

### Biomechanical Optimized Neural Engineering System (BONES)

### Overview of BONES as the Core Intelligence Layer of Autonomous Orthopedics (AO)

Autonomous Orthopedics (AO) systems described herein are enabled by an artificial intelligence architecture referred to as the Biomechanical Optimized Neural Engineering System (BONES).

BONES comprises one or more physics-informed artificial intelligence models configured to predict real-time biomechanical response of skeletal tissues and implant interfaces to applied mechanical forces.

Unlike conventional machine learning models that rely solely on empirical data fitting, BONES integrates fundamental biomechanical principles, material behavior models, and structural dynamics into neural network architectures.

BONES thereby provides real-time predictive intelligence governing force application, adaptive control, and safety thresholds in AO.

In preferred embodiments, BONES operates as the primary decision-making layer within the closed-loop force control architecture connecting OCSI instruments to robotic actuation.

### Physics-Informed Neural Network Architecture

BONES may be implemented using physics-informed neural networks (PINNs) trained on: biomechanical finite element simulations of bone-implant interactions; material property distributions across patient populations; experimental force-displacement datasets; and intraoperative sensor data collected by OCSI instruments.

The PINN architecture embeds governing equations of mechanics including: elastic and viscoelastic deformation models; fracture mechanics thresholds; frictional contact mechanics; impact dynamics; and vibrational response behavior.

Training constrains neural network outputs to obey physical laws, ensuring: predictive stability; physiologically realistic responses; and improved generalization across patient variability.

BONES thereby anticipates skeletal response rather than merely reacting to sensor feedback.

### Patient-Specific Biomechanical Modeling

BONES dynamically generates patient-specific biomechanical response profiles based on: real-time sensor measurements from OCSI instruments; preoperative imaging data when available; inferred material property estimations; and historical surgical outcome data.

These profiles may include: effective bone modulus maps; fracture risk envelopes; optimal interference fit ranges; allowable force vectors and magnitudes; and deformation tolerance thresholds.

Accordingly, AO applies customized force strategies for each patient rather than fixed procedural parameters.

### Real-Time Predictive Force Control

During orthopedic procedures, BONES continuously predicts the mechanical consequences of incremental force application.

For each control cycle, BONES may compute: projected deformation response; anticipated implant stability gain; proximity to fracture thresholds; expected interface micromotion; and optimal continuation or termination of force application.

These predictions are fed to the robotic control layer to: modulate applied force magnitude; adjust insertion velocity; modify force direction; initiate vibratory assistance; and halt force application when optimal stability is achieved.

Thus, AO transitions from reactive sensing to predictive force intelligence.

### Accelerated Computing for Real-Time Biomechanical Intelligence

In preferred embodiments, BONES is executed on accelerated computing hardware including: graphics processing units (GPUs); field programmable gate arrays (FPGAs); dedicated neural processing units (NPUs); or hybrid high-performance computing architectures.

Accelerated computation enables: millisecond-scale biomechanical predictions; real-time closed-loop force adaptation; and high-dimensional sensor data fusion.

Such performance is essential for high-force orthopedic procedures where mechanical responses evolve rapidly.

### Continuous Learning and System Refinement

BONES may incorporate continual learning mechanisms wherein intraoperative data collected by AO systems are used to: refine biomechanical models; improve predictive accuracy; adapt to rare anatomical conditions; and optimize surgical force strategies over time.

Data may be aggregated across surgical centers to build large-scale biomechanical intelligence networks while maintaining patient privacy.

This continuous refinement progressively enhances AO performance and safety.

### Safety Constraints and Predictive Risk Mitigation

Because BONES integrates physical law constraints, it inherently enforces safety envelopes that prevent: excessive force application; unstable deformation trajectories; fracture-inducing stress concentrations; and over-impaction leading to aseptic loosening.

AO systems thereby operate within predictive biomechanical safety margins rather than relying solely on reactive force thresholds.

### BONES as the Enabling Engine of Surgical Autonomy

Through predictive biomechanical intelligence, BONES transforms orthopedic robotics from: position-guided tools into force-intelligent autonomous systems.

BONES enables: standardized orthopedic assembly processes; patient-specific adaptive force application; reduction of surgical variability; and progressive automation of force-driven surgical tasks.

Accordingly, BONES constitutes the core artificial intelligence engine through which Autonomous Orthopedics (AO) achieves real-time adaptive and autonomous control.

### Closed-Loop Force Control and Progressive Autonomy in Autonomous Orthopedics (AO)

### Closed-Loop Cyber-Physical Force Regulation Architecture

Autonomous Orthopedics (AO) systems described herein operate as closed-loop cyber-physical control systems in which mechanical force application is continuously regulated through real-time sensing, biomechanical prediction, and robotic actuation.

The closed-loop architecture comprises: 1. OCSI sensorized instruments that generate real-time mechanical interaction data; 2. biomechanical estimation modules that quantify tissue response parameters; 3. BONES predictive artificial intelligence that forecasts mechanical consequences of force application; and 4. robotic actuators that execute controlled force adjustments.

At each control cycle, AO performs: acquisition of force and interface data; estimation of biomechanical properties; prediction of mechanical outcomes via BONES; and adaptive modification of applied force.

This loop repeats continuously throughout bone preparation and implant installation, enabling dynamic real-time force optimization.

### Force Vector Modulation and Multi-Dimensional Control

AO systems regulate not only the magnitude of applied force but also its directional vector, temporal profile, and rate of application.

Force control may include modulation of: axial compression forces; lateral alignment forces; torsional rotational forces; vibratory energy superposition; and impact pulse shaping.

BONES predicts multi-dimensional mechanical response allowing AO to: correct implant trajectory in real time; minimize asymmetric stress concentrations; optimize interface conformity; and improve fixation uniformity.

Such multi-axis force intelligence enables precise mechanical assembly beyond human sensorimotor capability.

### Assistive Force-Intelligent Surgical Mode

In certain embodiments, AO operates in an assistive mode wherein: OCSI instruments measure real-time mechanical interactions; BONES predicts biomechanical response; and AO provides force guidance to the surgeon via visual, auditory, or haptic feedback.

The system may display: optimal force ranges; warning thresholds; predicted stability outcomes; and recommended continuation or termination of force application.

In this mode, the surgeon retains direct physical control while benefiting from real-time biomechanical intelligence.

### Semi-Autonomous Adaptive Force Execution Mode

In further embodiments, AO operates in a semi-autonomous mode wherein robotic actuators directly apply force under BONES control while the surgeon supervises the procedure.

In this mode: AO autonomously modulates force magnitude and direction; adjusts insertion speed; applies vibratory assistance when advantageous; and halts force application at predicted optimal fixation thresholds.

The surgeon may intervene or override at any time.

Semi-autonomous operation enables standardized precision while preserving clinical oversight.

### Fully Autonomous Orthopedic Assembly Mode

In still further embodiments, AO executes fully autonomous force-driven surgical tasks.

Such tasks may include: bone cavity sizing and preparation; controlled broaching; implant insertion and seating; fixation optimization; and real-time alignment correction.

BONES governs all force decisions while robotic actuators execute mechanical operations without manual input.

Safety constraints embedded in BONES and closed-loop feedback enforce predictive biomechanical risk boundaries throughout autonomous execution.

### Dynamic Termination of Force Application

A critical function of AO systems is intelligent termination of force application based on biomechanical optimization rather than arbitrary thresholds.

Termination criteria may include: predicted maximal implant stability achieved; proximity to fracture risk envelopes; diminishing stability gain per force increment; and optimal interference fit saturation.

BONES continuously evaluates termination conditions, enabling AO to stop force application at the precise moment of optimal fixation.

### Learning-Based Optimization of Surgical Force Strategies

AO systems may continuously refine force strategies using reinforcement learning, supervised learning, and physics-constrained adaptation.

Through repeated procedures, BONES may: identify optimal force trajectories for specific anatomical profiles; improve prediction of failure thresholds; personalize surgical protocols; and minimize procedural variability.

Over time, AO develops increasingly precise biomechanical intelligence.

### Force Standardization and Surgical Reproducibility

By governing orthopedic assembly through closed-loop predictive force control, AO establishes standardized mechanical execution independent of individual surgeon experience.

Procedures become: reproducible; quantifiable; optimized; and scalable.

This enables consistent outcomes across practitioners and institutions.

### Networked Biomechanical Data Learning and Continuous Intelligence Evolution

### Distributed Surgical Data Acquisition

Autonomous Orthopedics (AO) systems described herein generate high-resolution biomechanical datasets during every surgical procedure.

Data may include, without limitation: force and torque profiles; displacement and deformation responses; vibration and acoustic signals; predicted versus actual biomechanical outcomes; implant stability metrics; termination conditions; and post-operative performance correlations.

These data are captured continuously by OCSI instruments and processed through BONES during closed-loop force control.

### Centralized and Federated Learning Architectures

In preferred embodiments, biomechanical data generated by AO systems may be transmitted to centralized or federated learning infrastructures.

Learning architectures may include: centralized cloud-based training platforms; distributed edge-learning systems; privacy-preserving federated learning models; and hybrid local-global optimization networks.

These architectures enable BONES models to be updated using aggregated surgical experience across multiple institutions without compromising patient privacy.

### Cross-Procedure Intelligence Accumulation

Each AO-enabled procedure contributes new biomechanical knowledge to BONES.

Accumulated intelligence may include: refined fracture risk envelopes; improved force-trajectory optimization; enhanced prediction of patient-specific variability; superior termination criteria; and adaptive vibration and impaction strategies.

Over time, BONES transitions from individual predictive models to population-scale biomechanical intelligence.

### Continuous Model Refinement and Deployment

Updated BONES models may be periodically or continuously deployed to AO systems in clinical use.

Deployment may include: incremental neural network weight updates; adaptive parameter tuning; real-time inference optimization; and safety-validated model versioning.

Thus, AO systems improve performance with each additional procedure performed globally.

### Learning Across Anatomical Regions and Procedures

Biomechanical knowledge generated in one surgical domain may be transferred to others.

For example: force-response patterns learned in hip arthroplasty may inform trauma fixation; cortical deformation models may inform spinal instrumentation; and vibratory insertion optimization may generalize across implant types.

BONES thereby evolves as a unified biomechanical intelligence engine across orthopedics.

### Predictive Outcome Optimization

Through large-scale data aggregation, BONES may correlate force strategies with: implant longevity; complication rates; fracture incidence; micromotion thresholds; and functional outcomes.

These correlations further refine force control strategies toward outcome-optimized surgical execution.

### Platform Evolution Analogous to Autonomous Vehicle Learning

Just as autonomous vehicles continuously improve by learning from millions of miles of driving data, AO systems improve by learning from millions of surgical force-interaction cycles.

Each procedure enhances BONES' predictive accuracy, safety margins, and optimization capability.

This continuous learning framework transforms AO from a static robotic tool into a self-improving biomechanical intelligence platform.

### Competitive and Technical Advantages of Networked Learning

The networked learning architecture enables: rapid intelligence scaling; continual safety enhancement; accelerated innovation; and persistent performance superiority over non-learning systems.

### Broad Surgical Applications and Embodiments of Autonomous Orthopedics (AO)

General Applicability Across Force-Driven Orthopedic Procedures The Autonomous Orthopedics (AO) systems described herein are applicable to any orthopedic procedure involving mechanical force interaction with bone, implants, or fixation devices.

AO is not limited to any specific anatomical region, implant type, or surgical technique.

Rather, AO governs the standardized adaptive application of force wherever bone is cut, shaped, compressed, impacted, expanded, vibrated, or mechanically engaged.

### Arthroplasty Procedures

In certain embodiments, AO is applied to joint replacement procedures including, without limitation: total hip arthroplasty; partial hip arthroplasty; revision hip arthroplasty; total knee arthroplasty; unicompartmental knee replacement; total shoulder arthroplasty; elbow replacement; and ankle replacement.

AO regulates bone preparation forces, implant seating forces, and fixation optimization for press-fit, cemented, or hybrid constructs.

### Trauma and Fracture Fixation

In further embodiments, AO is applied to trauma procedures including: intramedullary nail insertion; fracture reduction and stabilization; compression plating; external fixation systems; and expandable fixation devices.

AO adaptively controls reaming forces, nail insertion forces, fracture compression magnitude, and fixation loading profiles to optimize stability while preventing bone damage.

### Spinal Procedures

AO may be applied to spinal interventions including: pedicle screw placement; rod insertion and tensioning; interbody cage insertion; vertebral augmentation; and deformity correction systems.

Force-adaptive regulation optimizes bone engagement, screw purchase, cage seating, and load distribution.

### Bone Defect Reconstruction and Grafting

In additional embodiments, AO governs mechanical interactions during: bone graft compaction; defect filling; scaffold implantation; porous implant insertion; and biologic augmentation procedures.

BONES predicts structural response to optimize graft density and mechanical integration.

### Dental and Craniofacial Applications

AO may further be applied to: dental implant insertion; maxillofacial reconstruction; cranial fixation systems; and orthodontic anchorage devices.
where controlled mechanical force determines osseointegration and stability.

### Fixation Modalities and Implant Interfaces

AO applies to all fixation strategies including: press-fit interference fixation; cemented fixation; threaded fixation; expandable anchoring systems; porous osseointegration surfaces; and hybrid mechanical-biologic fixation.

Force regulation adapts dynamically based on interface behavior.

### Multi-Implant and Complex Construct Assembly

AO systems may coordinate force application across multiple implants or components including: modular joint systems; multi-level spinal constructs; fracture fixation assemblies; and patient-specific implant systems.

BONES predicts cumulative mechanical interactions to optimize overall construct integrity.

### Robotic Integration Modalities

AO may be implemented as: standalone robotic surgical systems; integrated modules within existing robotic platforms; instrument-mounted autonomous units; retrofitted smart-tool systems; or distributed sensor-actuator networks.

Thus, AO encompasses both dedicated autonomous robots and hybrid robotic-tool ecosystems.

### Universal Force-Intelligent Surgical Platform

Through its predictive force-adaptive architecture, AO establishes a universal orthopedic assembly platform applicable wherever mechanical force governs surgical success.

Embodiments of the present invention may thereby transcend specific procedures and constitutes a foundational mechanical intelligence system for orthopedic surgery.

### Safety, Validation, and Regulatory Control Frameworks

### Predictive Biomechanical Safety Envelopes

Autonomous Orthopedics (AO) systems described herein operate within dynamically generated biomechanical safety envelopes produced by BONES.

These envelopes define allowable ranges of: force magnitude; deformation response; stress concentration; impact energy; vibratory amplitude; and displacement progression, for each patient-specific anatomical configuration.

BONES continuously updates safety envelopes in real time based on sensor feedback and predicted mechanical response.

Force application outside of predicted safe boundaries is automatically prevented.

### Real-Time Risk Detection and Mitigation

AO systems monitor mechanical interaction patterns to detect emerging risk conditions including: excessive stress gradients; unstable deformation trajectories; approaching fracture thresholds; abnormal interface slippage; and unexpected mechanical resonance.

Upon detection of risk indicators, AO may: immediately reduce applied force; adjust force direction; initiate vibratory assistance; pause the procedure; or terminate force application entirely.

These responses occur autonomously within closed-loop control cycles.

### Redundant Sensor Architectures

In preferred embodiments, OCSI instruments incorporate redundant sensing modalities to ensure reliability and fault tolerance.

Redundancy may include: multiple independent force sensors; cross-validation between mechanical and electrical measurements; fusion of vibration, acoustic, and torque data; and diagnostic self-check routines.

Discrepancies between sensing channels trigger safety fallback modes.

### Human-in-the-Loop and Override Controls

AO systems support surgeon oversight and intervention at all autonomy levels.

Safety controls may include: manual override mechanisms; emergency stop interfaces; adjustable autonomy thresholds; and real-time transparency displays of force predictions and decisions.

Surgeons may transition between assistive, semi-autonomous, and autonomous modes at any time.

### Verification and Calibration Procedures

AO systems may perform automatic calibration routines prior to and during surgery.

Calibration may include: sensor zeroing; force response validation; actuator performance checks; and biomechanical model alignment.

Continuous validation ensures accuracy of force measurement and control.

### Regulatory Compliance Architecture

AO systems are designed to support regulatory requirements including: traceability of decision logic; recording of force profiles; audit trails of autonomous actions; explainable AI outputs grounded in physical models; and version control of BONES models.

These features enable certification, post-market surveillance, and clinical validation.

### Fail-Safe and Degradation Modes

In the event of component failure, data inconsistency, or model uncertainty, AO systems transition to safe operational states including: force limitation mode; manual control reversion; restricted autonomy operation; and procedural pause states.

This ensures patient safety under all contingencies.

### Continuous Safety Improvement Through Learning

Safety envelopes and risk models may be continuously refined using networked learning described herein.

Complication data, near-miss events, and mechanical anomalies inform BONES updates to further reduce risk probability.

### Representative Operational Workflows and Use Scenarios

### Force-Adaptive Bone Preparation Workflow

In one representative embodiment, AO governs bone cavity preparation using sensor-integrated ESSOB instruments.

The workflow may include: 1. insertion of an ESSOB-equipped reamer or broach into bone; 2. real-time acquisition of force, torque, vibration, and resistance data; 3. biomechanical estimation of bone stiffness and deformation response; 4. BONES prediction of cavity expansion behavior and fracture risk; 5. adaptive modulation of cutting force and rotational speed; and 6. termination of bone preparation at predicted optimal cavity dimensions.

Throughout the process, AO continuously updates force strategies to accommodate patient-specific bone response.

### Predictive Implant Insertion and Fixation Workflow

In another embodiment, AO controls implant installation using APIM and/or VIOI instruments.

The workflow may include: 1. positioning of the implant at the prepared cavity entrance; 2. initiation of controlled insertion via robotic actuators; 3. real-time force measurement at the bone-implant interface; 4. BONES prediction of stability gain versus fracture risk; 5. dynamic adjustment of insertion force magnitude and rate; 6. optional superposition of vibratory assistance; and 7. termination of insertion at predicted optimal fixation point.

AO thereby achieves standardized press-fit without reliance on manual impaction.

### Real-Time Alignment Correction Workflow

During insertion, AO may detect asymmetric resistance indicating misalignment.

In response, AO may: modify force vectors to correct implant trajectory; adjust insertion angle; apply lateral stabilization forces; and reduce insertion rate until alignment stabilizes.

This ensures optimal mechanical seating without excessive stress.

### Adaptive Fracture Fixation Workflow

In trauma applications, AO may control fracture reduction and fixation.

The workflow may include: 1. sensing resistance during reaming and nail insertion; 2. predicting cortical deformation behavior; 3. regulating compression forces across fracture sites; and 4. optimizing fixation tension for stability without bone damage.

AO thereby personalizes fixation mechanics to each fracture geometry.

### Progressive Autonomy Workflow

AO may transition through autonomy levels during a single procedure.

For example: assistive mode during initial bone preparation; semi-autonomous force control during implant insertion; and autonomous termination at optimal fixation.

Surgeons may supervise and intervene as desired.

### Learning-Enhanced Workflow Optimization

Over successive procedures, BONES refines predictions and force strategies.

Later workflows may achieve: faster preparation times; reduced force variability; improved fixation consistency; and lower complication risk.

Thus AO evolves with accumulated surgical experience.

### Multi-Tool Coordinated Workflow

AO may coordinate multiple OCSI instruments simultaneously.

For example: ESSOB preparing cavities while APIM prepares insertion parameters; VIOI optimizing vibratory profiles during seating; and real-time data fusion across instruments.

This enables holistic mechanical optimization.

### Emergency and Safety Response Workflow

If risk indicators emerge, AO initiates protective responses including: force reduction; procedural pause; surgeon alert; and controlled withdrawal of instruments.

Safety is maintained continuously.

### Additional Embodiments, Variations, and System Configurations

### Modular and Scalable System Architectures

AO systems may be implemented in modular configurations wherein sensing, artificial intelligence, and actuation subsystems are separable or integrable depending on clinical application.

Embodiments may include: instrument-mounted intelligence modules; centralized robotic control units; distributed sensor-actuator networks; portable autonomous surgical platforms; and integration within existing robotic systems.

Modules may be added, removed, or upgraded without altering core functionality.

### Hardware Variations

OCSI instruments may employ a wide range of sensor technologies including: piezoresistive sensors; optical fiber strain sensors; capacitive force sensors; ultrasonic transducers; electromagnetic sensors; and microelectromechanical systems (MEMS).

Actuation may include: electric motors; pneumatic systems; hydraulic actuators; piezoelectric drivers; and magnetic actuation.

### Artificial Intelligence Variations

BONES may be implemented using: physics-informed neural networks; hybrid physics-data-driven models; reinforcement learning controllers; Bayesian predictive models; and digital twin biomechanical simulators.

Multiple AI models may operate concurrently with ensemble decision fusion.

### Alternative Sensing and Estimation Methods

Biomechanical estimation may utilize: direct force measurement; indirect electrical inference; vibration signature analysis; acoustic emission monitoring; optical deformation sensing; and impedance spectroscopy.

These modalities may be combined through sensor fusion.

### Force Application Modalities

AO may regulate force via: continuous press-fit insertion; controlled pulsed impaction; harmonic vibratory assistance; progressive expansion systems; adaptive torque-driven insertion; and multi-axis stabilization forces.

### Local and Remote Operation

AO systems may operate: locally within surgical theaters; under remote supervision; with teleoperated override; and within hybrid autonomous-manual environments.

### Integration With Preoperative Planning Systems

AO may interface with imaging systems including: CT and MRI scans; fluoroscopy; navigation platforms; and patient-specific surgical plans.

Preoperative data may initialize biomechanical models used by BONES.

### Implant-Specific Optimization Profiles

AO may store implant-specific mechanical models allowing: customized force strategies per implant geometry; surface texture optimization; interference fit tuning; and stability prediction per design.

### Outcome-Based Adaptive Protocols

BONES may adjust force strategies based on: long-term implant performance data; revision history; and patient recovery metrics.

This enables outcome-driven surgical optimization.

The embodiments described herein are illustrative and not limiting.

Any configuration incorporating real-time force sensing, predictive biomechanical modeling, adaptive force control, and robotic or electromechanical actuation may be used in accordance with the systems and methods described herein, whether implemented as a standalone surgical instrument, a modular subsystem integrated into an existing robotic platform, a distributed sensor-actuator network, or a coordinated surgical control system operating across one or more orthopedic instruments.

### Patient-Specific Biomechanical Digital Twin Architecture

In some embodiments, the systems and methods described herein incorporate a patient-specific biomechanical digital twin configured to represent skeletal anatomy, material properties, and mechanical response to applied surgical force.

The biomechanical digital twin comprises a computational representation of bone geometry, density distribution, stiffness, and boundary conditions associated with an orthopedic procedure. The digital twin is configured to predict mechanical response of skeletal tissue, including deformation, stress distribution, fracture risk, and fixation stability, under applied force.

In preferred embodiments, the digital twin is initialized using preoperative patient data, including medical imaging and inferred biomechanical properties. During surgery, the digital twin is iteratively updated using real-time mechanical interaction data acquired from sensorized surgical instruments, including force, torque, vibration, and impedance signals.

The updated digital twin is used by the force-adaptive control system to generate predictive force strategies and safety envelopes governing mechanical execution of surgical tasks.

Force application is dynamically adjusted based on predicted response of the digital twin, including predicted fracture thresholds and stability progression.

In some embodiments, the fidelity of the biomechanical digital twin increases progressively as additional intraoperative data are acquired, enabling increasingly accurate personalization of force control throughout the procedure.

### Autonomous Surgical Control Orchestration Layer

In some embodiments, the systems described herein further comprise an autonomous surgical control orchestration layer configured to coordinate force-adaptive control, biomechanical prediction, safety governance, and multi-instrument execution.

The orchestration layer operates as a supervisory control architecture managing interaction among multiple functional modules, including a force control kernel, a biomechanical prediction module, a safety envelope enforcement module, an autonomy level management module, and a human override interface.

The orchestration layer is configured to manage transitions between assistive, semi-autonomous, and fully autonomous operation modes based on predicted biomechanical risk, procedural state, and system confidence. Safety constraints are enforced continuously by restricting force execution to predicted safe operating regions.

In embodiments involving multiple surgical instruments, the orchestration layer coordinates force execution across instruments such that mechanical actions by one instrument inform or constrain force application by another, thereby enabling synchronized and cooperative surgical execution.

In some embodiments, biomechanical control models and force strategies are stored, selected, and deployed across surgical systems under control of the orchestration layer. Execution data may be recorded to evaluate performance, update predictive models, and verify compliance with safety constraints.

The system and methods above have been described in general terms as an aid to understanding details of preferred embodiments of the present invention. In the description herein, numerous specific details are provided, such as examples of components and/or methods, to provide a thorough understanding of embodiments of the present invention. Some features and benefits of the present invention are realized in such modes and are not required in every case. One skilled in the relevant art will recognize, however, that an embodiment of the invention can be practiced without one or more of the specific details, or with other apparatus, systems, assemblies, methods, components, materials, parts, and/or the like. In other instances, well-known structures, materials, or operations are not specifically shown or described in detail to avoid obscuring aspects of embodiments of the present invention.

One or more components of each embodiment of the present invention may be implemented using a programmable computer system (general or specialized) which may include an AI general or special purpose computer, with models trained using data as described herein.

In an AI general-purpose computer, additional components to a general purpose computer (memory, processor, and interface elements) and specialized hardware are incorporated to handle the immense computational demands of training and running large language models. Alongside the standard CPU, these systems often include powerful graphics processing units (GPUs) and tensor processing units (TPUs) designed for parallel processing of large datasets and complex mathematical operations. GPUs and TPUs enable efficient training of AI models by accelerating matrix multiplications and other key operations. High-capacity and high-speed memory systems, such as HBM (High Bandwidth Memory), ensure rapid data transfer and storage during model training. Moreover, AI-specific hardware accelerators, like neural network processors and dedicated AI chips, further optimize performance and energy efficiency.

During the training of large language models, data is fed into the system, where it undergoes preprocessing to clean and structure it for the model. The training process involves iteratively adjusting the model's parameters by minimizing a loss function through backpropagation and gradient descent. The model retrieves instructions and data from memory, processes them through layers of artificial neurons, and updates its weights based on the calculated errors. This cycle repeats millions of times until the model converges to an optimal state. Once trained, the AI model can generate human-like text, understand context, and perform various language-related tasks. These advanced systems leverage parallelism, hardware acceleration, and vast computational power to handle the complexity and scale required for modern AI applications.

Development of one or more embodiments of the present invention may be developed in phases—Phase 1: Biomechanical Sensor Integration which involves full development of the electromechanical prototypes ESSOB, APIM, VIOI, capable of measuring bone resistance, acoustic feedback “chatter”, and kinematic and dynamic responses to force application; Phase2. AI-Augmented Decision Support (AI-ADS) which involves developing Machine Learning algorithms that infer optimal reaming and implant seating; and include key technologies such as natural language processing NLP, statistical modeling, Reinforcement learning (RL) for adaptive decision making in dynamic environments, Explainable AI (XAI) for transparent decision logic, and Edge computing, for real-time distributed decision-making, and Finite Element Analysis (FEA); Phase3. Real-Time Adaptive Robotics (Closed-loop AI force regulation); which involves transitioning from passive force measurement to active force regulation using closed-loop AI feedback systems; Phase 4. FDA Regulatory Pathway (510(k) premarket notifications); Phase 5. Clinical Trials & Surgeon Adoption (Proving improved outcomes); and Phase 6. Market Penetration & Scalability (Data-sharing networks for AI refinement).

Future impact of one or more embodiments of the present invention may include one or more of:

Potential advantages and future implementation pathways of one or more embodiments of the present invention may include one or more of the following.

In some embodiments, the systems and methods described herein may reduce reliance on purely manual orthopedic instruments by providing force-intelligent surgical tools configured to measure, estimate, and regulate mechanical interaction between orthopedic instruments, implants, and bone.

For example, conventional manually operated reamers, broaches, impactors, mallets, insertion handles, and related orthopedic instruments may be supplemented, modified, or replaced by sensor-integrated electromechanical instruments configured to generate real-time mechanical interaction data.

Such instruments may include force, torque, vibration, acoustic, electrical, displacement, or kinematic sensing elements, and may communicate with processing systems configured to estimate bone response, interface conditions, implant stability, fracture risk, or other biomechanical parameters relevant to orthopedic assembly.

In some embodiments, the disclosed systems may enhance real-time surgical augmentation by providing quantitative feedback, predictive recommendations, or adaptive control during bone preparation, implant insertion, fixation, alignment correction, or other force-driven orthopedic tasks.

Accordingly, a surgeon may be assisted by real-time information regarding applied force, resistance, implant seating progression, predicted stability, proximity to safety thresholds, and recommended continuation, modification, or termination of force application.

In further embodiments, robotic or electromechanical actuators may dynamically modulate reaming, broaching, impaction, vibratory insertion, compression, torque application, or other mechanical actions based on sensor feedback and predictive biomechanical modeling.

By converting qualitative tactile, auditory, and experiential judgments into measurable and repeatable control parameters, embodiments of the invention may improve reproducibility of orthopedic procedures across surgeons, institutions, implant systems, and patient anatomies.

In some embodiments, the disclosed systems may enable less-experienced or lower-volume surgeons to perform selected force-driven surgical tasks with increased quantitative guidance, thereby reducing dependence on subjective sensorimotor judgment alone.

The disclosed systems may also support progressive levels of automation, including force-aware decision support, surgeon-supervised semi-autonomous force execution, and autonomous execution of defined orthopedic assembly tasks within predetermined biomechanical safety boundaries.

In some embodiments, reductions in excessive force application, insufficient fixation, uncontrolled impaction, implant micromotion, intraoperative fracture, malalignment caused by uncontrolled force vectors, or other mechanically mediated complications may reduce revision burden, post-operative morbidity, or downstream healthcare costs.

In further embodiments, the disclosed systems may enable new clinical, operational, or economic models in which orthopedic value is associated not only with implant design or implant material, but also with the quality, reproducibility, and quantitative control of the surgical assembly process.

For example, procedure-specific force profiles, safety envelopes, implant-specific control parameters, and accumulated biomechanical interaction data may be used to standardize surgical execution, refine predictive models, support surgeon training, inform quality assurance, or improve future surgical planning.

Accordingly, embodiments of the present invention may provide a foundation for force-intelligent and progressively autonomous orthopedic surgery, in which sensor-integrated instruments, predictive biomechanical models, and closed-loop actuation systems cooperate to improve precision, consistency, safety, and scalability of orthopedic procedures.

It will also be appreciated that one or more of the elements depicted in the drawings/figures can also be implemented in a more separated or integrated manner, or even removed or rendered as inoperable in certain cases, as is useful in accordance with a particular application.

Additionally, any signal arrows in the drawings/Figures should be considered only as exemplary, and not limiting, unless otherwise specifically noted. Combinations of components or steps will also be considered as being noted, where terminology is foreseen as rendering the ability to separate or combine is unclear.

The foregoing description of illustrated embodiments of the present invention, including what is described in the Abstract, is not intended to be exhaustive or to limit the invention to the precise forms disclosed herein. While specific embodiments of, and examples for, the invention are described herein for illustrative purposes only, various equivalent modifications are possible within the spirit and scope of the present invention, as those skilled in the relevant art will recognize and appreciate. As indicated, these modifications may be made to the present invention in light of the foregoing description of illustrated embodiments of the present invention and are to be included within the spirit and scope of the present invention.

Thus, while the present invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the present invention. It is intended that the invention not be limited to the particular terms used in following claims and/or to the particular embodiment disclosed as the best mode contemplated for carrying out this invention, but that the invention will include any and all embodiments and equivalents falling within the scope of the appended claims. Thus, the scope of the invention is to be determined solely by the appended claims.

1-173. (canceled)

174. A force-adaptive orthopedic surgical control system comprising:
at least one orthopedic instrument configured to mechanically engage bone, an orthopedic implant, or an orthopedic fixation device during an orthopedic procedure;
one or more sensors operatively coupled to the orthopedic instrument and configured to generate real-time mechanical interaction data representative of interaction between the orthopedic instrument and the bone, orthopedic implant, or orthopedic fixation device;
a processing system configured to estimate, from the real-time mechanical interaction data, one or more biomechanical properties of skeletal tissue or a bone-implant interface;
a predictive biomechanical modeling subsystem configured to predict a mechanical response of the skeletal tissue or bone-implant interface to a candidate force application based at least in part on the one or more biomechanical properties;
a robotic or electromechanical actuation system configured to apply mechanical force to the orthopedic instrument, orthopedic implant, or orthopedic fixation device; and
a closed-loop control architecture configured to generate a force-control command for the robotic or electromechanical actuation system based on the real-time mechanical interaction data and the predicted mechanical response,
wherein the force-control command regulates at least one of magnitude, direction, rate, temporal profile, impact energy, vibratory energy, torque, displacement, or termination of the mechanical force during a force-mediated orthopedic assembly task.

175. The system of claim 174, wherein the real-time mechanical interaction data comprise at least one of force, torque, vibration, acoustic response, deformation, displacement, electrical impedance, motor current, motor voltage, rotational resistance, cutting resistance, insertion resistance, or impact response.

176. The system of claim 174, wherein the orthopedic instrument comprises at least one of a reamer, broach, impactor, insertion handle, drill, threaded fixation instrument, vibratory insertion device, implant insertion device, intramedullary nail insertion device, fracture fixation instrument, or robotic end-effector.

177. The system of claim 174, wherein the one or more biomechanical properties comprise at least one of effective stiffness, elasticity, compliance, bone density, estimated bone density, or a parameter correlated with bone density, interface friction, contact condition, interference fit, deformation response, implant seating progression, micromotion risk, or fracture threshold proximity.

178. The system of claim 174, wherein the predicted mechanical response comprises at least one of predicted deformation, predicted stability gain, predicted fracture risk, predicted implant seating progression, predicted micromotion, predicted interface conformity, or predicted termination condition.

179. The system of claim 174, wherein the predictive biomechanical modeling subsystem comprises:
an input interface configured to receive the real-time mechanical interaction data from the one or more sensors;
one or more computational models configured to predict skeletal tissue response or bone-implant interface response to applied force;
a real-time inference engine configured to generate the predicted mechanical response based at least in part on the real-time mechanical interaction data; and
an output interface configured to provide at least one of the predicted mechanical response, a predicted stability gain, a predicted biomechanical risk, or data used by the closed-loop control architecture to generate the force-control command,
wherein the one or more computational models comprise one or more physics-informed models trained or parameterized using biomechanical simulation data, empirical mechanical interaction data, intraoperative sensor data, or combinations thereof.

180. The system of claim 174, wherein the orthopedic instrument comprises an Electronic Signature Sizing of Bone instrument configured to generate an electronic signature associated with bone cavity preparation.

181. The system of claim 180, wherein the electronic signature is used to regulate at least one of reaming force, broaching force, cutting force, rotational speed, advancement rate, instrument size selection, or termination of bone preparation.

182. The system of claim 174, wherein the orthopedic instrument comprises an Automatic Prosthesis Installation Machine configured to regulate insertion, seating, impaction, or press-fit fixation of an orthopedic implant.

183. The system of claim 182, wherein the closed-loop control architecture terminates insertion, seating, or impaction based on at least one of predicted implant stability, predicted fracture risk, predicted interface condition, predicted seating progression, or predicted reduction in incremental stability gain.

184. The system of claim 174, wherein the orthopedic instrument comprises a Vibratory Insertion of Orthopedic Implants system configured to apply controlled vibratory energy during implant insertion or seating.

185. The system of claim 184, wherein the force-control command regulates one or more vibration parameters including amplitude, frequency, duration, or timing relative to insertion force.

186. The system of claim 174, wherein the closed-loop control architecture regulates a force vector direction in addition to force magnitude.

187. The system of claim 174, wherein the system is selectively operable in at least two of: a force-aware assistive mode, a semi-autonomous force execution mode, and an autonomous force execution mode.

188. The system of claim 187, further comprising a user interface configured to permit surgeon supervision, adjustment, authorization, or override of force execution.

189. The system of claim 174, wherein the closed-loop control architecture enforces a biomechanical safety envelope based on at least one of maximum force, maximum torque, deformation limit, displacement limit, vibration limit, impact energy limit, predicted fracture threshold, predicted instability condition, or model uncertainty or system confidence.

190. The system of claim 189, wherein the system transitions to a restricted force mode, manual mode, paused mode, or terminated mode when the predicted mechanical response is within a predetermined margin of, reaches, or exceeds the biomechanical safety envelope.

191. A method of performing force-adaptive orthopedic assembly, the method comprising:
mechanically engaging bone, an orthopedic implant, or an orthopedic fixation device using a sensor-integrated orthopedic instrument;
acquiring real-time mechanical interaction data from the sensor-integrated orthopedic instrument;
estimating one or more biomechanical properties of skeletal tissue or a bone-implant interface from the real-time mechanical interaction data;
predicting, using a predictive biomechanical model, a mechanical response to a candidate force application;
generating a force-control command based on the predicted mechanical response; and
applying mechanical force using a robotic or electromechanical actuator according to the force-control command,
wherein the force-control command is updated during the orthopedic assembly task based on additional real-time mechanical interaction data.

192. The method of claim 191, further comprising modifying or terminating force application when the predicted mechanical response indicates that a stability condition, safety threshold, fracture threshold, micromotion threshold, seating condition, or threshold corresponding to diminishing stability gain per force increment has been reached.

193. The method of claim 191, wherein acquiring real-time mechanical interaction data comprises acquiring at least one of force, torque, vibration, acoustic response, electrical impedance, motor current, motor voltage, displacement, or insertion resistance.

194. The method of claim 191, wherein estimating one or more biomechanical properties comprises estimating at least one of stiffness, compliance, interference fit, fracture threshold proximity, implant seating progression, or micromotion risk.

195. The method of claim 191, wherein applying mechanical force comprises at least one of continuous press-fit insertion, controlled pulsed impaction, controlled vibratory insertion, adaptive torque-driven insertion, or multi-axis stabilization.
